(19) United States
(12) Patent Application Publication
(10) Pub. No.: US 2013/0197397 A1
(43) Pub. Date: Aug. 1, 2013

(54) Methods and Assessment Scales for Measuring Wrinkle Severity

(71) Applicant: REVANCE THERAPEUTICS, INC., Newark, CA (US)

(72) Inventors: Jacob M. Waugh, San Francisco, CA (US); Andrew Blitzer, New York, NY (US)

(73) Assignee: REVANCE THERAPEUTICS, INC., Newark, CA (US)

(21) Appl. No.: 13/751,124

(22) Filed: Jan. 27, 2013

Related U.S. Application Data
(60) Provisional application No. 61/591,451, filed on Jan. 27, 2012.

(51) Int. Cl. A61B 5/00; A61B 5/107

(52) U.S. Cl. CPC A61B 5/442; A61B 5/1072; A61B 5/4839
USPC 600/587

(57) ABSTRACT

This invention provides devices, methods, and assessment scales for assessing the severity of wrinkles. Wrinkle assessment using the devices, methods, and assessment scales of the invention can be performed in a clinical setting for the purpose of simply characterizing a wrinkle, or as part of a treatment regimen that varies according to the severity of the wrinkle. Thus, this invention also provides methods of reducing the appearance of wrinkles, and kits comprising for evaluating and treating wrinkles.

### FIELD OF THE INVENTION

This invention relates to methods and assessment scales for assessing the level of severity of wrinkles.

### BACKGROUND OF THE INVENTION

Wrinkles are familiar effects of aging. Treatments to alleviate different characteristics of wrinkles have various results depending on the level of severity of wrinkles.

The age related progression of wrinkles has been previously described and categorized as a sequential progression of stages by Glogau, in “Aesthetic and anatomic analysis of the aging skin.” Semin. Cutan. Med. Surg. 1996; 15(3): 134-138. For instance, for subjects in their mid-teens, lateral canthal lines (LCL), which emanate from the distal corner of the eye, and are known as crow's feet, occur naturally during smile, but are absent at rest. Such lateral canthal lines reflect a positive emotional connection that is not viewed to be a sign of aging. Patients typically seek treatment only after they have lateral canthal lines when their facial muscles are at rest, as such lines typically result from aging. Lateral canthal lines at rest have been shown to be a major factor in the perception of facial age.

A popular cosmetic method to treat wrinkles involves the administration of botulinum toxin. Exemplary administration methods are by single or multiple injections of the toxin into a patient, or by topical application, as described in U.S. application Ser. No. 11/072026, which is hereby incorporated by reference. Botulinum toxin type A (BoNTA) blocks cholinergic neurotransmission by preventing acetylcholine release at peripheral neuromuscular junctions. Local injections of BoNTA are effective for a temporary improvement of facial lines. Such facial lines include glabellar lines, which form between the eyebrows and above the nose; and lateral canthal lines (LCL).

Previous wrinkle treatments lack reliable wrinkle measurement devices to assess wrinkle severity. In addition, treatment of wrinkles has generally been based on the subjective determinations of the treating physician, rather than on a standard treatment protocol tailored to the severity of the wrinkle. Accordingly, there is a need for improved methods of assessing wrinkles and corresponding methods of treating wrinkles.

### SUMMARY OF THE INVENTION

This invention provides devices, methods, and assessment scales for assessing the severity of wrinkles. Wrinkle assessment using the devices, methods, and assessment scales of the invention can be performed for the purpose of simply characterizing a wrinkle, or as part of a treatment regimen that varies according to the severity of the wrinkle. Thus, this invention also provides methods of reducing the appearance of wrinkles, and kits comprising for evaluating and treating wrinkles.

In one aspect, this invention provides a wrinkle length measurement device. The device includes a handle and a measurement section connected to the handle. The measurement section includes measurement units that originate at an interior portion of the measurement section and extend distally towards an edge of the measurement section. The measurement units, taken together, form a measurement scale.

In another aspect, the invention also provides an assessment scale for assessing wrinkle severity. The assessment scale has two or more levels, wherein each level corresponds to a different degree of wrinkle severity. Each degree of wrinkle severity is defined based on a combination of at least two measured physical characteristics of a wrinkle, which can be, for example, wrinkle length and wrinkle depth.

In another aspect, this invention provides an assessment system for assessing wrinkle severity. The assessment system includes a measuring device to evaluate wrinkle length. The measuring device may include a handle and a measurement section attached to the handle. The measurement section includes measurement units that originate at an interior portion of the measurement section and extend distally towards an edge of the measurement section to form a calibrated device. The assessment system also includes an assessment scale for assessing wrinkle severity. The assessment scale has a plurality of levels, wherein each level corresponds to a different degree of wrinkle severity. Each degree of wrinkle severity is defined based on a combination of at least two measured physical characteristics of a wrinkle.

In yet another aspect, the invention provides a method for assessing wrinkle severity. The method includes measuring wrinkle length and assessing wrinkle severity according to an assessment scale. The wrinkle length may be measured with any calibrated device suitable for measuring wrinkle length. Optionally, a wrinkle length measurement device as described herein is used to measure wrinkle length. For instance, the wrinkle length measurement device may be with a device that includes a handle and a measurement section connected to the handle. In such a device, the measurement section may be calibrated by including measurement units that originate at an interior portion of the measurement section and extend distally towards an edge of the measurement section. The measurement units, taken together, form a calibrated measurement scale. Optionally, the units of the measurement scale are calibrated to correspond with severity levels of an assessment scale. The assessment scale may include a plurality of levels, where each level corresponds to a different degree of wrinkle severity, as described herein.

The invention also provides a method for reducing the appearance of wrinkles in a subject. The method includes determining the length of a wrinkle, assessing wrinkle severity according to an assessment scale, and treating the wrinkle in accordance with a treatment that corresponds to the level of severity of the wrinkle to reduce the appearance of the wrinkle. The length of a wrinkle can be determined using a wrinkle length measurement device as described herein. The assessment scale includes a plurality of levels, with each level corresponding to a different degree of wrinkle severity, as described herein. Each degree of wrinkle severity is defined based on a combination of wrinkle length and at least one other measured physical characteristic of a wrinkle, such as wrinkle depth.

In one aspect, the invention provides a kit that includes a wrinkle length measurement device and a medium that comprises an assessment scale. The wrinkle length measurement device includes a handle and a measurement section with measurement units as described herein. The kit further includes an assessment scale as described herein.

### BRIEF DESCRIPTION OF THE FIGURES

FIG. 1: shows a measuring device to evaluate the length of lateral canthal lines (LCL). FIG. 1A shows a view of one side of the device, where measurement units on a measurement section are clearly visible. FIG. 1B shows a view of the other side of the device, which optionally has instructions printed thereon.

FIG. 2 shows a measuring device to evaluate the length of lateral canthal lines (LCL).

### DETAILED DESCRIPTION OF THE INVENTION

The term “wrinkle”, as used herein, refers to a fold or crease in the skin. Wrinkles can vary in size and intensity, from fine lines to deep furrows. Wrinkles in skin may be classified into three different types: dynamic wrinkles, static wrinkles and wrinkle folds. Dynamic wrinkles are caused by repeated contractions of muscles underlying the skin. For example, frowning or furrowing causes wrinkles between the eyebrows (i.e., glabellar lines), while smiling and/or squinting causes wrinkles at the distal corners of the eyes (i.e., lateral canthal lines). Static wrinkles, or wrinkles at rest, when the face is in a neutral or natural position, result from a loss of elasticity in skin, which may arise from a variety of factors, including sun damage, poor nutrition, smoking, and genetic factors, or from spasms or tones of muscles. Wrinkle folds, which may appear as deep grooves between the nose and mouth, for example, arise from the sagging of underlying facial structure.

Certain commonly observed wrinkle patterns may result from a combination of static and dynamic wrinkles. For example, and without wishing to be bound to any theory, it is believed that lateral canthal lines (LCL) at smile arise from the contractions of several muscles, such as zygomaticus major, orbicularis oculi, levator anguli, oris major, levator anguli, oris minor. It is believed, however, lateral canthal lines at rest arise only from the spasm or tone of orbicularis oculi.

Generally, the methods of this invention are suitable for measurement and assessment of wrinkle severity of all types of wrinkles. Optionally, the wrinkles are evaluated when the underlying muscles are at rest. This invention also provides for treatment decisions based on the degree of wrinkle severity as determined according to the invention. Generally speaking, the invention is suitable for assessing and treating wrinkles present on any area of skin of a subject in need of treatment. Non-limiting examples of areas that may be treated include the face, head, neck, hands, feet, shoulders, chest, torso and back. In addition, when the area to be evaluated is the face, the wrinkles may be located in specific subregions of the face, such as the forehead, eyes, temples, cheeks, or jawline.

In one aspect, this invention provides a device for measuring wrinkle length. The invention recognizes that while measuring the length of everyday objects is generally straightforward, the measurement of wrinkles, particularly on the face, poses certain technical challenges. For example, most people do not like to have foreign objects, such as a ruler, pressed against their face and may involuntarily move their head during the measurement. The measurement of lateral canthal lines (i.e., crow's feet wrinkles”) is especially difficult. Lateral canthal lines emanate from the outer corners of the eyes and fan outward across the temple. To measure such lines with a conventional ruler, one is faced with two undesirable alternatives. In one method, the origin of a ruler (i.e., the “zero”) is placed at the corner of the eye, and the ruler is aligned with each lateral canthal line to make the measurement. This method is dangerous, because the origin of a ruler is typically at a corner, which is usually sharp. Placing a sharp corner of a ruler near a subject's eye could damage the eye if the clinician or the subject makes a sudden unexpected movement. Alternatively, the origin of the ruler can be aligned with the end of the lateral canthal line that is distal to the corner of the eye. However, in this configuration, the body of the ruler presses against the eyelid of the subject. The pressure exerted by a ruler against the eyelid is uncomfortable to the subject.

Accordingly, this invention provides devices for measuring the length of wrinkles. Generally, the devices contemplated by the invention are designed to avoid the dangers and discomfort associated with using a conventional ruler to measure wrinkles on a subject's face. For example, in certain preferred embodiments, the wrinkle length measurement devices do not have any sharp corners. Rather, any corners that otherwise would be present are rounded to minimize the potential for damage to the eye in case the device accidentally contacts the eye. In addition, in certain embodiments, the origin of the measurement scale is located at an interior portion of the device, rather than at one of the ends of the device. In this way, it is less likely that a sudden unexpected movement by the clinician or subject will result in an eye injury caused by one of the ends of the device.

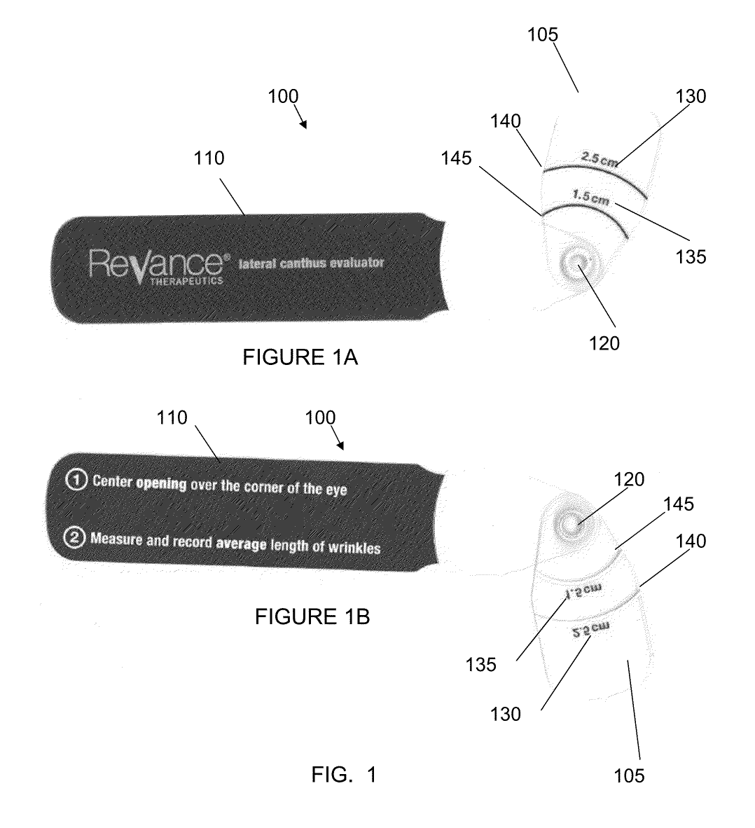

FIG. 1 shows one implementation of a wrinkle length measurement device that is consistent with the principles of the invention. As shown in FIG. 1A, wrinkle length measurement device 100 comprises a handle 110 and measurement section 105, which optionally may be fabricated from a transparent material, such as plastic, for reasons discussed herein. The handle 110 is pivotally connected to measurement section 105 at junction 120, such that measurement section 105 can be rotated about junction 120 when handle 110 is fixed. Junction 120 also serves as an origin for a measurement scale on measurement section 105. The measurement scale further includes marks 140 and 145, which may be curved as shown in this embodiment and which correspond to measurement units 130 and 135, respectively. In this embodiment, measurement units 130 and 135 are annotated with the markings “1.5 cm” and “2.5 cm” to indicate that marks 140 and 145 have radii of curvature that are 1.5 cm and 2.5 cm, respectively, with respect to the center of junction 120. The curved gradations facilitate the measurement of multiple lateral canthal lines originating from essentially a single point (the corner of the eye) and radiating outward at different angles. Junction 120 serves as the origin of the measurement scale on measurement section 105.

FIG. 1B shows the back side of the wrinkle length measurement device shown in FIG. 1A. In this embodiment, text is printed directly onto handle 110 to provide instructions for using wrinkle length measurement device 100.

In certain preferred embodiments, wrinkle length measurement device 100 is used to measure the length of a subject's lateral canthal lines. Typically, a user will hold onto handle 110 and use it to position junction 120 over the corner of the subject's eye. Measurement section 105 is then rotated until it overlaps with the subject's lateral canthal lines. In this embodiment, since measurement section 105 is made of a transparent material, a user can see all of the lateral canthal lines through measurement section 105 and compare their lengths to gradations 140 and 145 in order to measure the lines. The transparent material permits the user to determine the length of all lateral canthal lines under measurement section 105 without repositioning measurement section 105. Furthermore, since handle 110 and measurement section 105 are pivotally connected, handle 110 can be positioned away from the subject's eyes even while measurement section 105 is positioned over a subject's lateral canthal lines during measurement. In this way, device 100 provides the subject with a safer and more comfortable measurement process. Optionally, the measuring portion is shaped to have curvature to approximate the side of the head around the edge of the eye. Such a curved measurement component would facilitate measuring line length as the extend from a source and continue of a curved surface. In addition, the measuring portion is optionally flexible, such that it can be conformed to the shape of the side of the head during the measurement process.

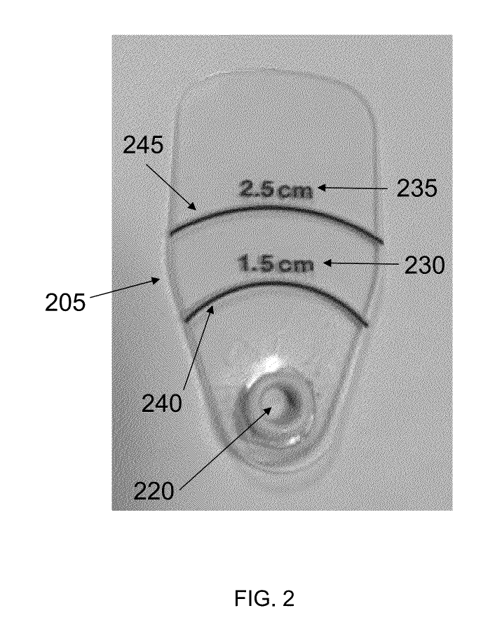

If desired, wrinkle length measurement device 100 may be fabricated without a handle 110. FIG. 2 shows a non-limiting example of this embodiment. In FIG. 2, wrinkle length measurement device 205 includes origin 220 which serves as the origin for a measurement scale that includes marks 240 and 245, which optionally may be curved and which correspond to measurement units 230 and 235, respectively. Measurement units 230 and 235 are annotated with the markings “1.5 cm” and “2.5 cm” to indicate that marks 240 and 245 have radii of curvature that are 1.5 cm and 2.5 cm, respectively, with respect to the center of origin 220. These curved gradations facilitate the measurement of multiple lateral canthal lines originating from essentially a single point (the corner of the eye) and radiating outward at different angles. When wrinkle length measurement device 205 is used to measure lateral canthal lines, origin 220 is typically positioned at the corner of the eye and the length of the lateral canthal line is evaluated by comparing it to marks 240 and 245. Optionally, wrinkle length measurement device 205 is made of a transparent material, so that a user can see all of the lateral canthal lines through wrinkle length measurement device 205 and compare their lengths to gradations 240 and 245 in order to measure the lines. The transparent material permits the user to determine the length of all lateral canthal lines under wrinkle length measurement device 205 without repositioning.

In certain embodiments, the wrinkle length measurement device is sterilized. For instance, when the wrinkle length measurement device is used for measuring wrinkles around the eye, the device optionally may be a single-use device that is sterilized during manufacturing and distributed in sterilized packaging. In this way, the spread of contagious eye diseases, such as bacterial or viral conjunctivitis, may be minimized. In another embodiment, the device may be made of a sterilizable material, non-limiting examples of which include plastics, metals, or combinations thereof. In such embodiments, an end-user, such as a clinician, may sterilize the device prior to use.

In another aspect, the invention provides an assessment scale for assessing the severity of a wrinkle. In certain embodiments, the invention provides a wrinkle assessment scale that is content valid, reliable, construct valid, able to detect clinical change, and able to establish a threshold for treatment benefit. In this context, an assessment scale is deemed to be “content valid” if it is developed based on the following two activities: (i) identification of relevant measured observables, for example by a review of the literature, clinician input and direct patient input (e.g., through interviews also known as “concept elicitation”); and (ii) demonstration that intended users can understand the assessment scale and what it is designed to measure (e.g., through clinical advisory board evaluation or structured interviews termed “cognitive debriefing” for subjects). An assessment scale is deemed “reliable” if the each of the observables upon which the assessment scale is based can be reproducibly measured. The reliability of an assessment scale may be established through high intra- and inter-observer correlation values. As is known in the art, kappa statistics may be used to assess concordance. Kappa values range between 0 (no agreement) and 1 (absolute agreement). As is known in the art, a kappa value in the range of <0.20 shows poor agreement; a kappa value in the range of 0.21-0.40 shows fair agreement; a kappa value in the 0.41-0.60 range shows moderate agreement; a kappa value in the 0.61-0.80 range shows good agreement; and a kappa value in the 0.81-1.00 range shows very good (near perfect) agreement. An assessment scale is deemed to be “construct valid” if can be shown that the assessment scale actually and reproducibly measures what it is designed to measure. As the skilled artisan will appreciate, establishing construct validity is often an important component of validation of a measurement scale. In certain embodiments, construct validity of an assessment scale may be established by showing high correlations to other scales measuring similar concepts. An assessment scale is deemed “able to detect change” if the chosen measured observables associated with the assessment scale permit a user to significantly and consistently distinguish changes due to treatment. An assessment scale is deemed “able to establish a threshold for treatment benefit” if a user can compare changes as a result of treatment to a pre-determined threshold in order to determine whether the threshold is met.

Generally, the assessment scales of the invention comprise a plurality of levels, where each level corresponds to a different degree of wrinkle severity. In certain embodiments, the levels of the assessment scale are defined by reference to one or more physical characteristics of the wrinkle, non-limiting examples of which include length, width, depth, area, morphology, position, skin rigidity, volume, shape of underlying muscle, quantity of wrinkles, and wrinkle-to-wrinkle distance. Each level in the assessment scale may be distinguished from the others based on descriptors that relate to measured physical characteristics. The form of the descriptors is not particularly limited and may comprise text, images, or combinations thereof. Optionally, a rating system may be used to uniquely identify each level of the assessment scale. For instance, the rating system may be numerical, with the lowest number of the rating system corresponding to the least severe level of wrinkles, and the highest number of the rating system corresponding to the most severe level of wrinkles. The use of combinations of assessment scales is also contemplated by the invention. For instance, an assessment scale characterizing wrinkle severity when a patient is at rest may be used in conjunction with an assessment scale that characterizes the wrinkle severity when the skin is under muscular tension (e.g., due to smiling, frowning, squinting, and the like). Two or more assessment scales can be used to arrive at an overall assessment, which can then be used as a basis for further evaluation or treatment, as disclosed herein.

In certain implementations, the invention provides an assessment scale that is specifically constructed for assessing the severity of lateral canthal lines. One aspect of this invention is the recognition that evaluation of at least two physical characteristics is necessary to construct a assessment scale for measuring the severity of lateral canthal lines that is content valid, reliable, construct valid, able to detect clinical change, and able to establish a threshold for treatment benefit. Another aspect of the invention is the recognition that an assessment scale for measuring the wrinkle severity, for example of lateral canthal lines, can be constructed by using just two physical characteristics, namely wrinkle length and wrinkle depth. Surprisingly, such an assessment scale involving just two physical characteristics is content valid, reliable, construct valid, able to detect clinical change, and able to establish a threshold for treatment benefit. Generally, the wrinkle length and wrinkle depth may be measured by any method known in the art. In measuring wrinkle length measurements may be made in fractions of inches centimeters or using any other arbitrary calibrated scale. Calibrations in millimeters or eighths or sixteenths of an inch provide a more sensitive ability to detect changes in wrinkle severity. In certain preferred embodiments, however, the wrinkle length is measured using the wrinkle length measurement device disclosed herein. The wrinkle depth may be measured by a variety of techniques, non-limiting examples of which include multi-photon microscopy, silicone casting/visiometry, laser profilometry and the like. Optionally, the wrinkle depth is determined by psychometric evaluations by a clinician. Such psychometric evaluations are well known in the art and can be conducted by using, without limitation, questionnaires, tests, assessments, and interviews. In one particular embodiment, wrinkle depth may be measured by clinicians using a questionnaire which requires the clinician to classify the wrinkle as “absent,” “shallow” or “deep.” Of course, other terms or a different number of terms may be used without departing from the spirit and scope of this invention. In addition, a wrinkle optionally may be assessed by touch to provide a qualitative evaluation of the wrinkle depth. For instance, a clinician may press down on a wrinkle directly or may run his or her finger perpendicularly to the wrinkle line. When the wrinkle and the surrounding skin form ridges that feel set and rigid, the wrinkle is typically characterized as a deep wrinkle. On the other hand, when the wrinkle and surrounding skin is soft and yields readily to a clinician's direct touch, the wrinkle typically is characterized as a shallow wrinkle. Qualitatively assessing wrinkle depth by touch may be used in combination with, or instead of, the physical measurements or psychometric measurements of wrinkle depth described herein.

In addition, a clinician may try to “spread open” a wrinkle to characterize the elasticity of the skin surrounding it. This evaluation may be accomplished manually, for example, by pressing one's fingers onto the skin on either side of the wrinkle and then moving the fingers apart, such that the skin is stretched. In such evaluations, it is to be understood that the degree to which the skin is stretched is such that the patient feels no pain and the skin tissue is not damaged. To the extent that the wrinkle appears wider as a result of such applied spreading forces, a clinician may conclude that the skin surrounding the wrinkle is sufficiently elastic that the wrinkle would respond favorably to treatment with a paralytic agent, such as botulinum toxin and the like. For example, a paralytic agent such as botulinum toxin may be administered if the width of the wrinkle at its widest point increases by at least 20%, 30%, 40% or 50% as a result of the applied spreading forces. On the other hand, if a wrinkle's width at its widest point increases by less than 20%, 15%, or 10% as a result of the applied spreading forces, the surrounding skin may not be sufficiently elastic for the wrinkle to respond favorably to the administration of a paralytic agent, and other treatments that do not involve administration of paralytics may be preferable. Such other treatments include, for example, surgery or the use of fillers, as is known in the art.

The form of the assessment scale is not particularly limited, and may be in any format suitable for storing and organizing information. For example, in certain embodiments, the assessment scale is stored electronically, such as on a computer readable medium or in a database (e.g., a relational database), and accessed as needed. In certain embodiments, the assessment scale is depicted as a table, where each row of the table corresponds to a different level of severity. Tables 1 and 2 show examples of assessment scales that are consistent with the principles of the invention. In particular, Table 1 shows an assessment scale for measuring the severity of lateral canthal lines when the facial muscles of a subject are at rest.

TABLE 1

| | Wrinkle | |
|---|---|---|
| Rating | Severity | |
| Score | at Rest | Description |
| 0 | Absent | No visible wrinkles |
| 1 | Minimal | Minimal wrinkles, within 1.5 cm radius of the |
| | | lateral canthus and may be minimally etched |
| 2 | Mild | Shallow wrinkles, extending between 1.5 to 2.5 cm |
| | | radius of the lateral canthus and may be minimally |
| | | etched |
| 3 | Moderate | Moderately deep wrinkles, extending between 1.5 to |
| | | 2.5 cm radius of the lateral canthus and moderately |
| | | etched |
| 4 | Severe | Very deep wrinkles, exceeding 2.5 cm radius of the |
| | | lateral canthus and may be deeply etched |

Included in Table 1 is a numerical rating system (from 0-4) as well as text describing each level in the assessment scale. In this case, each level is defined by reference to both wrinkle length and wrinkle depth, which are determined using methods as described herein.

Table 2 shows an assessment scale for measuring the severity of lateral canthal lines when a subject is smiling. The assessment scale in Table 2 also includes a numerical rating system (from 0-4) and text describing each level of the assessment scale. In this exemplary embodiment, however, only one physical characteristic (i.e., the shape of the underlying lateral orbicularis oculi muscles) is used to characterize each level in the assessment scale. Also shown in Table 2 is an exemplary five-tier scale for psychometric evaluation of lateral canthal lines, in this case using the terms “absent,” “minimal,” “mild,” “moderate,” and “severe.”

TABLE 2

| Rating | Orbicularis | |
|---|---|---|
| Score | Activity at Smile | Description |
| 0 | Absent | No visible muscle bulging of the orbicularis |
| 1 | Minimal | Minimal muscle bulging of the orbicularis |
| 2 | Mild | Mild muscle bulging of the orbicularis |
| 3 | Moderate | Moderate muscle bulging of the orbicularis |
| 4 | Severe | Prominent muscle bulging of the orbicularis |

In yet another aspect, the invention provides a method for assessing wrinkle severity. The method includes a step of measuring at least one physical characteristic of a wrinkle and assessing wrinkle severity according to an assessment scale. Optionally, the method includes measuring two physical characteristics of a wrinkle, or optionally more than two physical characteristics of a wrinkle. The one or more measured physical characteristics can include any physical characteristic disclosed herein. Preferably, one of the physical characteristics is wrinkle length or the shape of the underlying muscle. The wrinkle length may be measured by any suitable method, including by use of the wrinkle length measurement device disclosed herein. The method further includes the step of assessing wrinkle severity according to an assessment scale comprising a plurality of levels, as disclosed herein. In certain embodiments, the levels of the assessment scale are defined by reference to wrinkle length and at least one other measured physical characteristic of a wrinkle, such as wrinkle depth. Optionally, the wrinkle also may be evaluated by spreading apart the wrinkle as described herein. For example, physical evaluation of a wrinkle by spreading it apart may be performed if the wrinkle rates as a “3” (moderate) or “4” (severe) on the scales set forth in Tables 1 and 2 above.

The invention also provides a method for reducing the appearance of wrinkles in a subject. The method comprises assessing a level of severity of a wrinkle, such as a lateral canthal line, using the methods described herein, and treating the wrinkle with a treatment that corresponds to the determined level of severity. In certain embodiments, wrinkle severity is assessed as part of an initial evaluation. Following the initial evaluation, a treatment protocol may be chosen according to the severity level of the wrinkle. The methods described herein for assessing wrinkle severity also may be used for sequential measurements, in order to assess progress and/or the outcome of the treatment over a period of time. The period of time may be, without limitation, any time sufficient to detect changes in the physical characteristics of the wrinkle, such as a change in wrinkle length or wrinkle depth. In certain embodiments, the time period may be one day, three days, one week, two weeks, three weeks, four weeks, six weeks, or eight weeks, six months, a year or according to a schedule established by anyone assessing the progress of treatment, including for example, the individual undergoing treatment, the physician or other health care professional.

In certain embodiments, once the level of severity of a wrinkle is determined, a treatment corresponding to the determined level of severity is administered. Generally, treatment involves administering an effective amount of an anti-wrinkle composition. The term “effective amount” as used herein means an amount of a composition that is sufficient to produce the desired effects, but that is implicitly safe amount (i.e. one that is low enough to avoid serious side effects). Desired effects include, but are not limited to, the attenuation of a physical characteristic of a wrinkle, such as a reduction in wrinkle length or wrinkle depth, for example.

Anti-wrinkle compositions contemplated by the invention are not particularly limited. For instance, the anti-wrinkle treatment optionally contains a chemodenervating agent, non-limiting examples of which include botulinum toxin, saxitoxin, tetanus toxin, tetrodotoxin and combinations thereof. In certain embodiments, the chemodenervating agent comprises one of the serotypes of botulinum toxin (viz., botulinum toxin type A, B, C₁, D, E, F, or G), which optionally may be present as an isolated neurotoxin. Anti-wrinkle compositions contemplated by the invention may also comprise other anti-wrinkle agents known in the art, non-limiting examples of which include retinol, alpha-hydroxy acid, collagen, elastin, and hyaluronic acid.

The anti-wrinkle compositions may be injected or topically administered. When the anti-wrinkle composition is administered by injection, the injection may be intradermal, intramuscular or subcutaneous. For example, in certain embodiments, an injectable anti-wrinkle composition comprising botulinum toxin is used with the methods and devices disclosed herein. Alternatively, the anti-wrinkle composition may be administered topically, using, for example, the compositions disclosed in W02008/045107, U.S. Pre-Grant Publication No. 20060182766, or U.S. Pre-Grant Publication No. 20070116724. In certain embodiments, the anti-wrinkle compositions include those disclosed in U.S. Pat. No. 7,807,780 or U.S. Pre-Grant Publication 20050196414. It is to be understood that the foregoing references, like all references cited herein, are incorporated by reference in their entirety.

In one aspect, the invention also provides a kit for assessing wrinkles. The kit may comprise a device for measuring at least one physical characteristic of a wrinkle, a non-limiting example of which is the wrinkle length measurement device disclosed herein. The kit optionally includes an assessment scale, which, without limitation, may be present in the kit as a table, chart, or as a data file on a computer-readable medium. The assessment scale comprises a plurality of levels, with each level corresponding to a different degree of wrinkle severity, as described herein. Optionally, the kit may include an anti-wrinkle composition or a series of anti-wrinkle compositions, which may be administered according to a level of wrinkle severity determined using the assessment scale. In certain embodiments, the kit contains one or more anti-wrinkle compositions that are administered as a single-dose treatment. Alternatively, in certain embodiments, the kits may include highly concentrated anti-wrinkle compositions that are diluted by the end user for use in multiple applications, for example.

### EXAMPLE 1

### A Lateral Canthal Line Assessment Scale

This example describes a clinical scale, called the “Investigator's Global Assessment of Lateral Canthal Line Severity Scale” (“IGA-LCL scale”), which was developed to assess lateral canthal lines in a resting neutral facial position. The IGA-LCL scale allows the direct evaluation of the action of a chemodenervating drug on the relevant target muscle, the orbicularis oculi muscle, and thus provides an appropriate and specific means of evaluating the drug.

The IGA-LCL scale was developed to be content valid, reliable, construct valid, able to detect clinical change, and able to establish threshold for treatment benefit. The IGA-LCL scale was refined and validated following several steps. First, concept elicitation was undertaken and content validity established. The identification of the important and relevant physical characteristics of a wrinkle for evaluating lateral canthal lines was based upon a literature review, clinician input and patient input. Surprisingly, only two physical characteristics, namely depth and length of the lateral canthal lines, consistently emerged as the central focus of physicians and patients upon considering the severity of lateral canthal lines. Thus, these physical characteristics became the basis of the quantitative scale development effort. A lateral canthus evaluator (LCE), as shown in FIG. 1, was employed to standardize length determination. Psychometric depth evaluations were made by experienced clinicians using visual inspection. The depth categories used were “shallow” and “deep.” Both length and depth were evaluated separately as part of the clinician assessment. The combination of the two attributes resulted in a unique, non-overlapping rating score, as illustrated in the assessment scale in Table 1. The attributes were organized to ensure that a subject's lateral canthal lines must improve in both length and depth in order to achieve a 2-point improvement from their baseline scores of moderate or severe.

To confirm the validity of the assessment method, traditional validation studies were undertaken to evaluate scale reliability through intra- and inter-observer correlations. Statistical estimates of consistency assess the degree of agreement between different individuals (inter-rater) and the reproducibility of response by the same individual (intra-rater). The evaluation of intra-rater reliability (the same rater on two different occasions) was based on the comparison of pre-treatment IGA-LCL scores recorded by trained investigators at two separate study visits two weeks apart on live subjects. Photographs were not used as a basis for these assessments. Kappa estimates of 0.89 and 0.88, based on the rating of 17 raters and 451 subjects, indicated very good intra-rater reliability. Additional studies were conducted to evaluate inter-rater reliability. The first study used two pairs of raters to evaluate 31 subjects. Kappa estimates for this study were 0.81. In the second study, eight physicians with experience in aesthetic outcomes individually assessed ten live models encompassing all ratings. All ratings were performed on live subjects. The overall weighted kappa estimates for this study were 0.77, confirming good to very good agreement between raters using the rating scores of Table 1.

Following identification and justification of the physical characteristics of the wrinkle proposed to be measured, the IGA-LCL scale was developed based upon clinician and patient response. Additionally, content validity of the IGA-LCL scale was established by clinician review, which confirmed that depth and length were central to clinical assessment of lateral canthal line severity. After confirmation of content validity, traditional validation studies were undertaken to evaluate scale reliability through intra- and inter-observer correlations. Kappa statistics were used to assess concordance for the IGA-LCL scale. Evaluation of intra-rater reliability (the same rater on two different occasions) was based on the comparison of screening and baseline severity assessments, as summarized in Table 3.

TABLE 3
Intra-rater Reliability of IGA-LCL Scale

| | | | | Number of | |
|---|---|---|---|---|---|
| | LCAs | Number of | Number of | Scores | |
| | Assessed | Exact | Scores | Differing | |
| Site | (N) | Matches | Differing by 1 | by 2 | Kappa† |
| 007 | 20 | 20 (100%) | 0 (0%) | 0 (0%) | 1.0000 |
| 008 | 78 | 76 (97.4%) | 2 (2.6%) | 0 (0%) | 0.9484 |
| 009 | 36 | 36 (100%) | 0 (0%) | 0 (0%) | 1.000 |
| 010 | 24 | 24 (100%) | 0 (0%) | 0 (0%) | 1.000 |
| 013 | 36 | 22 (61.1%) | 14 (38.9%) | 0 (0%) | 0.2500 |
| 014 | 108 | 105 (97.2%) | 3 (2.8%) | 0 (0%) | 0.9423 |
| 016 | 60 | 60 (100%) | 0 (0%) | 0 (0%) | 1.000 |
| Overall | 362 | 343 (94.8%) | 19 (5.2%) | 0 (0%) | 0.8945 |

†Weighted and unweighted Kappa were identical.

Based upon kappa estimates of 0.89 and 0.88, there was very good intra-rater reliability demonstrated across a total of 17 raters and 451 subjects. Subjects enrolled in these studies had moderate or severe wrinkles at the beginning of the study, at baseline. The results demonstrated a very good agreement within raters in all studies that implemented the IGA-ICL scale. Two further studies were conducted to evaluate inter-rater reliability. The first study used two pairs of raters to evaluate 31 subjects. Based upon kappa estimates of 0.81, a second study was undertaken, with a larger number of participating investigators. This study allowed eight physicians with experience in aesthetic outcomes to individually evaluate ten live models encompassing all ratings on the IGA-LCL scale. All ratings were performed on live subjects. The overall weighted kappa estimates for this study were 0.77, confirming good to very good agreement between raters using the IGA-LCL (see Table 4).

TABLE 4
Inter-rater Reliability of IGA-LCL Scale (RT001-MK001)

| | | Number of | Number of | Number of | | |
|---|---|---|---|---|---|---|
| | LCAs | Exact | Scores | Scores | | Weighted |
| Site | Assessed | Matches | Differing by 1 | Differing by 2 | Kappa | Kappa |
| 007/009 | 16 | 14 (87.5%) | 2 (12.5%) | 0 (0%) | 0.8261 | 0.9126 |
| 013 | 16 | 6 (37.5%) | 10 (62.5%) | 0 (0%) | 0.2271 | 0.5855 |
| 014 | 16 | 11 (68.8%) | 4 (25.0%) | 1 (6.3%) | 0.6117 | 0.7405 |
| 009 | 16 | 13 (81.3%) | 3 (18.8%) | 0 (0%) | 0.7333 | 0.8696 |
| 015 | 16 | 7 (43.8%) | 9 (56.3%) | 0 (0%) | 0.2727 | 0.5955 |
| 016 | 16 | 15 (93.8%) | 1 (6.3%) | 0 (0%) | 0.9179 | 0.9592 |
| 010 | 16 | 14 (87.5%) | 2 (12.5%) | 0 (0%) | 0.8333 | 0.9116 |
| 017 | 16 | 7 (43.8%) | 9 (56.3%) | 0 (0%) | 0.2727 | 0.5814 |
| Overall | 128 | 87 (68.0%) | 40 (31.3%) | 1 (0.8%) | 0.5795 | 0.7717 |

Once appropriate reliability was established, other required measurement properties were evaluated, such as construct validity. Construct validity and clinical relevance were demonstrated by confirming that the IGA-LCL scale is directly related to patient-based measures of lateral canthal lines, including patients' self-perception of improvement and severity. The patient was the sole driver for treatment and thus defined the clinical meaningfulness and importance of a result in this indication. In this context, “clinically meaningful” was defined by the condition to be addressed, which in this case was a baseline severity in lateral canthal lines (in a neutral facial position) for which a patient seeks improvement. The investigator scale in an aesthetic indication provided objectivity and clinical validation of the patient's own outcome. Since the IGA-LCL scale is the most empirically designed and objective scale of its type, the increments and results were clinically meaningful based upon patient responses. Thus the results of the evaluation were patient based outcomes.

Given the importance of patient-based outcome measures, correlations between the investigator-rated IGA-LCL scale and the responses on a patient-reported psychometric outcome scale, called the “Patient Global Impression of Change” (PGIC) were used to evaluate construct validity in two clinical trials. PGIC was a psychometric evaluation based on a 7-point scale (much improved, improved, a little improved, no change, a little worse, worse, much worse). Correlations in severity scores between the IGA-LCL scale and a patient self-rated static score of severity, known as the “Patient Severity Assessment” (PSA), were also examined PSA, which was also a psychometric evaluation, mirrored the IGA-LCL as a 5 point scale (absent, minimal, mild, moderate and severe). The patient reported outcomes, PGIC and PSA, were both developed and tested through the in-depth interviews with 31 patients who had never been treated with botulinum toxin. Both psychometric scales encompassed similar concepts to the IGA-LCL and thus represented appropriate benchmarks for clinical relevance and construct validity.

The correlations between the scores for the IGA-LCL scale and PSA were examined in two studies. In both studies, there was a positive relationship between the two instruments. The IGA-LCL scale demonstrated substantial agreement with PSA scores (right side: kappa=0.80 and left side: kappa=0.76). Furthermore, when IGA-LCL scale results were correlated with patient-reported assessments of PGIC improvement in LCL severity, both Spearman and Pearson correlation coefficients showed a statistically significant agreement between the IGA-LCL and the PGIC scales (r=0.3317 to r=0.3972, p=0.048 to p=0.0006 for Pearson correlation; r=0.3697 to r=0.4673; p=0.027 to p<0.0001 for Spearman correlation).

Through use of the assessment scale and methods of the present invention, a tested Botulinum Toxin Type A Topical Gel as disclosed, for example, in U.S. Pre-Grant Publication No. 20050196414 was demonstrated to meet pre-determined criteria for treatment effectiveness of lateral canthal lines. Subjects were required to have bilateral (both eyes) lateral canthal lines graded as either moderate (3) or severe (4) at rest based on the severity scale (ratings of 0-4 as detailed in Table 1). Patients received 0.5 mL of Botulinum Toxin Type A Topical Gel or control applied to each lateral canthal area (LCA) for 30 minutes; a non-adhesive occlusive dressing was utilized to ensure that patients did not inadvertently transfer the drug during the dwell time. A cleansing step was used after the dwell time to remove and inactivate residual Botulinum Toxin Type A Topical Gel.

In general, the results indicated that when clinicians evaluated a positive change in lateral canthal lines, patients also perceived improvement in their lateral canthal lines. Likewise, when physicians reported no change, lower levels of patient-reported improvements were also observed. The clinical relevance of improvement on IGA-LCL scale was confirmed by a traditional anchor-based approach which correlated the IGA-LCL scale to the anchor of Global Patient-Reported Measure as a standard for aesthetic outcome. Correlation between IGA-LCL and PGIC change was extremely high with Spearman correlations of r=0.70 for right eye IGA change to PGIC (P<0.0001) and r=0.73 for left eye IGA change to PGIC (P<0.0001). Responders at “Improved, or Much Improved” on PGIC had 2 point or greater bilateral IGA improvement at the selected RT001 dose in 80% of subjects. Thus, clinical relevance by improvement on the validated PGIC corresponded in the majority of subjects with improvement on IGA-LCL. The pattern and magnitude of the Spearman correlations between the scores for the IGA-LCL and a subject rating of severity measuring a similar concept (PSA) were also studied. There was the expected positive relationship between the two instruments.

Similarly, improvement as assessed by IGA-LCL scale and by a Patients' Global Impression of Change (PGIC) scale were closely related in both studies as well. Thus, the IGA-LCL scale showed positive correlations with both patient-based instruments measuring a similar concept, thus supporting construct validity and clinical relevance.

After establishment of construct validity by comparing the IGA-LCL scale to the PGIC and PSA scales, the IGA-LCL scale was evaluated for ability to detect change. The ability to detect change can be evaluated by looking at pre-/post-treatment changes. The ability of the IGA-LCL to detect change was prospectively examined in two Phase 2 studies. Specifically, Spearman correlations were calculated for the change from pre-treatment to the week 4 follow up visit in the IGA-LCL Severity Scale. All comparisons were statistically significant (P<0.0001) and strong in magnitude with an r>0.60.

In summary, in the context of treatment, the IGA-ICL scale showed change and was correlated with the wrinkle severity of each patient. The IGA-LCL scale discriminated treatment effect reliably and with notably low placebo rates, as summarized in Table 5.

TABLE 5
Number and Percentage of Lateral Canthal Areas with Improvement in
Lateral Canthal Line Severity at Rest from Baseline

| | | RT001 25 ng/mL | Control | |
|---|---|---|---|---|
| | Improvement | % Improvement | % Improvement | |
| Day | on IGA-LCL | (n = 136) | (n = 132) | P-value |
| 28 | ≧1 point | 103 (75.7%) | 29 (22.0%) | <0.0001 |
| 28 | ≧2 point | 70 (51.5%) | 14 (10.6%) | <0.0001 |

P-value from CMH

Sensitivity to change (treatment response) in the IGA-LCL scale was characterized by its ability to generate scores that reflect actual changes in lateral canthal line severity. Significant 1 point or greater and, separately, significant 2 point or greater improvement was observed on the IGA-LCL scale across both studies for RT001 versus controls. Improvement on the IGA-LCL scale was shown to be reliable, clinically meaningful, sensitive and statistically robust as an endpoint in comparison between RT001 at various doses and across time-points versus controls.

The results also demonstrate that by using an anchor-based approach to evaluate the severity scores on the IGA-LCL, patients report the physical characteristics of their wrinkles as “improved” or “much improved.” The average change in rating score for patients reporting being ‘improved’ on the PGIC supports a change of −2 in their rating score.

These scores established the level of change that represents a threshold for clinically meaningful benefit. The change in rating scores on the IGA-LCL at which patients reported being improved at all (“a little improved” or better) were evaluated. Table 6, below, shows that the average change score on individual left (−1.00) and right IGA-LCL (−1.00) for patients reporting being ‘a little improved’ on the PGIC. Table 7, which summarizes the proportion of patients at each level of change on the PGIC and each of the LGA-LCL scale, supports a change of −1 as showing a clinically important level improvement.

TABLE 6
PGIC Score Relationship at Week 4 with IGA-LCL Change Scores Between
Baseline and Week 4

| | IGA-LCL Change Scores (Left) | | | | IGA-LCL Change Scores (Right) | | | |
|---|---|---|---|---|---|---|---|---|
| PGIC | 0 | 1 | 2 | 3 | 0 | 1 | 2 | 3 |
| Much Worse | 0 (0.0%) | 0 (0.0%) | 0 (0.0%) | 0 (0.0%) | 0 (0.0%) | 0 (0.0%) | 0 (0.0%) | 0 (0.0%) |
| Worse | 0 (0.0%) | 0 (0.0%) | 0 (0.0%) | 0 (0.0%) | 0 (0.0%) | 0 (0.0%) | 0 (0.0%) | 0 (0.0%) |
| A Little Worse | 0 (0.0%) | 0 (0.0%) | 0 (0.0%) | 0 (0.0%) | 0 (0.0%) | 0 (0.0%) | 0 (0.0%) | 0 (0.0%) |
| No Change | 30 (34.1%) | 10 (11.4%) | 4 (4.5%) | 0 (0.0%) | 25 (28.4%) | 14 (15.9%) | 5 (5.7%) | 0 (0.0%) |
| A Little Improved | 6 (6.8%) | 4 (4.5%) | 6 (6.8%) | 0 (0.0%) | 5 (5.7%) | 6 (6.8%) | 5 (5.7%) | 0 (0.0%) |
| Improved | 0 (0.0%) | 4 (4.5%) | 10 (11.4%) | 1 (1.1%) | 0 (0.0%) | 3 (3.4%) | 11 (12.5%) | 1 (1.1%) |
| Much Improved | 0 (0.0%) | 0 (0.0%) | 8 (9.1%) | 5 (5.7%) | 0 (0.0%) | 0 (0.0%) | 9 (10.2%) | 4 (4.5%) |

| | | |
|---|---|---|
| Spearman | R = 0.73, p = <.0001 | r = 0.70, p = <.0001 |
| Correlation | | |

TABLE 7
IGA-LCL Mean Change Scores
from Baseline to Week 4 by PGIC Response

| | | IGA | |
|---|---|---|---|
| | IGA (Left Side) | (Right Side) | Mean of IGA |
| | Mean Change | Mean Change | Mean Change |
| | Score | Score | Score |
| PGIC Group | 4 Weeks | 4 Weeks | 4 Weeks |
| Much Improved | −2.38 (N = 13) | −2.31 (N = 13) | −2.345 (N = 26) |
| Improved | −1.80 (N = 15) | −1.87 (N = 15) | −1.835 (N = 30) |
| A Little | −1.00 (N = 16) | 1.00 (N = 16) | −1.000 (N = 32) |
| Improved | | | |
| No Change | −0.41 (N = 44) | −0.55 (N = 44) | −0.480 (N = 88) |
| A Little Worse | (N = 0) | (N = 0) | (N = 0) |
| Worse | (N = 0) | (N = 0) | (N = 0) |
| Much Worse | (N = 0) | (N = 0) | (N = 0) |
| Overall p-value | <.0001 | <.0001 | <.0001 |

All references, including patent applications and publications cited herein, are incorporated by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. An assessment scale for assessing wrinkle severity, the assessment scale comprising
a plurality of levels, wherein each level corresponds to a different degree of wrinkle severity, and
wherein each degree of wrinkle severity is defined based on a combination of at least two measured physical characteristics of a wrinkle.

2. The assessment scale according to claim 1, wherein each degree of wrinkle severity is defined based on a combination of two measured physical characteristics of a wrinkle.

3. The assessment scale according to claim 2, wherein the two measured physical characteristics are wrinkle length and wrinkle depth.

4. The assessment scale according to claim 1, wherein one of the at least two measured physical characteristics of a wrinkle is wrinkle length.

5. The assessment scale according to claim 1, wherein one of the at least two measured physical characteristics of a wrinkle is wrinkle depth.

6. The assessment scale according to claim 1, wherein each level in the assessment scale is represented by text, images, or a combination thereof describing a magnitude of each of the at least two measured physical characteristics.

7. The assessment scale according to claim 1, wherein the assessment scale is in tabular form.

8. The assessment scale according to claim 1, wherein the assessment scale is stored electronically.

9. The assessment scale according to claim 1, wherein the wrinkle is selected from the group consisting of a lateral canthal line, a glabellar line, a forehead line, a platysma line, a nasolabial line, or a perioral line.

10. The assessment scale according to claim 9, wherein the wrinkle is a lateral canthal line.

11. A method for reducing the appearance of wrinkles in a subject, the method comprising:
determining the length of a wrinkle;
assessing wrinkle severity according to an assessment scale, wherein the assessment scale comprises a plurality of levels, each level corresponding to a different degree of wrinkle severity, and each degree of wrinkle severity being defined based on a combination of wrinkle length and at least one other measured physical characteristic of a wrinkle, and
treating the wrinkle in accordance with a treatment that corresponds to the level of severity of the wrinkle to reduce the appearance of the wrinkle.

12. The method according to claim 11, wherein the wrinkle is selected from the group consisting of a lateral canthal line, a glabellar line, a forehead line, a platysma line, a nasolabial line, or a perioral line.

13. The method according to claim 12, wherein the wrinkle is a lateral canthal line.

14. The method according to claim 11, wherein the step of treating the wrinkle comprises administering an effective amount of an anti-wrinkle composition.

15. The method according to claim 14, wherein the anti-wrinkle composition is administered by injection.

16. The method according to claim 14, wherein the anti-wrinkle composition is administered by topical application.

17. The method according to any one of claims 14-16, wherein the anti-wrinkle composition comprises a chemodenervating agent selected from the group consisting of botulinum toxin, saxitoxin, tetanus toxin, tetrodotoxin and combinations thereof

18. The method according to claim 17, wherein the chemodenervating agent comprises botulinum toxin.

19. The method according to claim 18, wherein the botulinum toxin is a botulinum neurotoxin.

20. A wrinkle length measurement device comprising
a measurement section comprising measurement units originating at an interior portion of the measurement section and extending distally towards an edge of the measurement section to form a measurement scale.

21. The wrinkle length measurement device according to claim 20, further comprising a handle connected to the measurement section.

22. The wrinkle length measurement device according to claim 21, wherein the handle and the measurement section are unitary.

23. The wrinkle length measurement device according to claim 22, wherein the handle and the measurement section are formed from a single piece of material.

24. The wrinkle length measurement device according to claim 21, wherein the handle and the measurement section are pivotally connected.

25. The wrinkle length measurement device according to claim 21, wherein the handle is connected to a region of the measurement section that is closer to the origin of the measurement scale than to the distal end of the measurement scale.

26. The wrinkle length measurement device according to claim 20 or 21, wherein the measurement section comprises a transparent material.

27. The wrinkle length measurement device according to claim 26, wherein each of the measurement units is indicated by a corresponding gradation, each gradation having a radius of curvature that is defined with respect to the origin of the measurement scale.

28. The wrinkle length measurement device according to claim 20, wherein the measurement section is calibrated to measure facial lines.

29. The wrinkle length measurement device according to claim 28, wherein the facial lines are lateral canthal lines.

30. The wrinkle length measurement device according to claim 20, wherein measurement section comprises at least two measurement units.

31. The wrinkle length measurement device according to claim 20, wherein the measurement section comprises measurement units expressed in terms of English or metric units of length.

32. The wrinkle length measurement device according to claim 20, wherein the device is configured to be a single-use device.

33. The wrinkle length measurement device according to claim 20, wherein the device is sterilized and enclosed in a sterilized container.

34. A method of evaluating a wrinkle, the method comprising
measuring the length of a wrinkle using the wrinkle length measurement device according to claim 20 or 21, and
optionally evaluating the elasticity of the skin surrounding the wrinkle by spreading the wrinkle apart; and
optionally evaluating the wrinkle depth by manually touching the wrinkle.

35. A kit comprising
a wrinkle length measurement device according to claim 20 or 21, and
an assessment scale, wherein the assessment scale comprises a plurality of levels, each level corresponding to a different degree of wrinkle severity, and each degree of wrinkle severity being defined based on a combination of wrinkle length and at least one other measured physical characteristic of a wrinkle.

36. The kit according to claim 35, wherein the at least one other measured physical characteristic of a wrinkle is wrinkle depth.

37. The kit according to claim 36, further comprising an anti-wrinkle composition.

38. The kit according to claim 37, wherein the anti-wrinkle composition comprises a chemodenervating agent selected from the group consisting of botulinum toxin, saxitoxin, tetanus toxin, tetrodotoxin and combinations thereof.

39. The method according to claim 38, wherein the chemodenervating agent comprises botulinum toxin.

40. The method according to claim 39, wherein the botulinum toxin is a botulinum neurotoxin.
